(19) United States
(12) Patent Application Publication
(10) Pub. No.: US 2011/0136108 A1
(43) Pub. Date: Jun. 9, 2011

(54) XBP1(S) Protein Acting as an Adipocyte Differentiation Marker Having a Facility to Regulate Differentiation into Adipocytes, and an Application Therefor

(75) Inventors: Oh Joo Kwon, Seoul (KR); Yoon Mi Cho, Seoul (KR)

(73) Assignee: CATHOLICE UNIVERSITY INDUSTRY ACADEMIC COOPERATION FOUNDATION, Seoul (KR)

(21) Appl. No.: 12/936,168

(22) PCT Filed: Apr. 3, 2009
(86) PCT No.: PCT/KR2009/001722

§ 371 Date: Feb. 22, 2011

(30) Foreign Application Priority Data
Apr. 3, 2008 (KR) 10-2008-0031028

(51) Int. Cl. C12Q 1/68; C07K 14/435; C07H 21/02; C12Q 1/02; C12N 15/12

(52) U.S. Cl. 435/6.1; 530/350; 536/24.5; 435/29; 536/23.5

(57) ABSTRACT

Disclosed are a protein marker, indicative of an increase in differentiation into adipocytes, comprising an XBP1(S) having an amino acid sequence of SEQ ID NO. 1, 2 or 3, and the uses thereof in developing a promoter of adipocyte differentiation and a method for promoting adipocyte differentiation, a repressor of adipocyte differentiation and a method for repressing adipocyte differentiation, an agent and a method for screening a repressor of adipocyte differentiation, and a method for reducing rosiglitazone's side effect of causing obesity. Also, provided is a protein marker, indicative of an increase in differentiation into adipocytes, comprising an XBP1(U) having an amino acid sequence of SEQ ID NO. 4, 5 or 6. When targeting the XBP1(S) gene or protein, a compound capable of blocking or restraining differentiation into adipocytes can be used to develop an agent for the prevention and treatment of obesity.

### TECHNICAL FIELD

The present invention relates to the regulation of differentiation into adipocytes. More particularly, the present invention relates to a protein marker, capable of indicating an increase in differentiation from pre-adipocytes, adipocyte precursor cells or stem cells into adipocytes, comprising as an active ingredient an XBP1(S) {X box-binding protein 1, spliced form} having an amino acid sequence of SEQ ID NO. 1, 2 or 3, and the use thereof in developing a promoter of adipocyte differentiation and a method for promoting adipocyte differentiation, a repressor of adipocyte differentiation and a method for repressing adipocyte differentiation, an agent and a method for screening a repressor of adipocyte differentiation, and a method for reducing rosiglitazone's side effect of causing obesity.

Also, the present invention is concerned with a protein marker, capable of indicating an increase in differentiation from pre-adipocytes, adipocyte precursor cells or stem cells into adipocytes, comprising as an active ingredient an XBP1(U) {X box-binding protein 1, unspliced form} having an amino acid sequence of SEQ ID NO. 4, 5 or 6.

### BACKGROUND ART

With the advance of society in economical terms, obesity is on the rise as one of the most serious diseases affecting society. WHO has finally declared obesity as a disease to be treated. Obesity is a metabolic disease resulting from an imbalance between intake and consumption of calories. From the morphological point of view, obesity occurs due to the hypertrophy or hyperplasia of adipose cells in the body. Obesity is one of the most common nutritional disorders in Western society, and is rapidly increasing in incidence rate in Asian countries which are adapting a westernized diet and lifestyle. Thus, the health policy of these countries is stressing the treatment and prophylaxis of obesity. Obesity not only disheartens individuals, but also is a major risk factor which increases the incidence of various adult diseases. Obesity is known to have a direct correlation with type II diabetes, hypertension, hyperlipidemia, cardiac diseases, etc. (Cell 87:377, 1999), and obesity-associated diseases including arteriosclerosis and cardiovascular disease are generically termed “metabolic syndrome” or “insulin resistance syndrome.” Because obesity increases the incidence rates of various metabolic diseases and weight loss decreases them, it can be inferred that adipocytes rich in fat mediate the relationship between obesity and metabolic disease.

Once considered only as an energy storage tissue which stores excessive energy in the form of triacylglycerol and releases energy when needed, adipose tissues are now accepted as an important endocrine organ for controlling energy homeostasis by secreting various adipokines including aiponectin, leptin and resistin (Trends Endocrinol Metab 13:18, 2002). Accordingly, the understanding of adipocyte proliferation and materials secreted from adipocytes and the examination of the in vivo regulation mechanism of the secreted materials are believed to be a basis on which effective agents for the treatment of various diseases associated with obesity can be developed. Active research is thus being conducted into the regulation of adipocyte differentiation. As concerns the increased adipose cells of obese patients, they are differentiated mainly from preadipocytes and precursor cells.

The endoplasmic reticulum (hereinafter referred to as the ER) is an intracellular organelle in which the metabolism of lipids, glycose, cholesterols and proteins are generally regulated. The ER is responsible for the production of proteins, in which various mechanisms including molecular chaperoning are performed to construct proteins of accurate structures. Hence, the ER is more developed in cells secreting lots of proteins than in the cells secreting a small quantity of proteins (Curr Opin Cell Biol 17:409, 2005). The ER is also where triglyceride droplets are formed (FEBS Lett 580:5484, 2006). The differentiation of preadipocytes into adipocytes requires greater amounts of differentiation-associated peptides and lipid mediators, so that the function of the ER responsible for servicing the increased demand is very important. In addition, the storage of a tremendous quantity of triacylglycerol therein imparts a great stress to the potential of the ER of obese individuals. As such, the environment in which the ER is burdened with extracellular factors is generically referred to as ER stress, and the response to surmount the ER stress is called unfolded protein response (hereinafter referred to as “UPR”).

ER stress beyond the limit of adaptation induces apoptosis, leading to cell death. Further, ER stress-induced apoptotic cell death is known to correlate with diabetes and other various obesity-associated diseases (Cardiovas Hematol Disord Drug Targets 7:205, 2007). Obesity induces ER stress (Science 306:457, 2004), and ER stress is in turn a link between obesity and insulin resistance (Nature 420:333, 2002; Proc Natl Acad Sci, U.S.A. 103:10741, 2006). On the one hand, there is evidence to show that the increased plasma free fatty acid levels of obese people play an important role in the induction of ER stress in pancreatic cells, thus developing diabetes therein (Endocrinology 147:3398, 2006; Diabetologia 50:752, 2007). The increase of intracellular triacylglycerol level found in obesity induces ER stress to kill insulin-secreting pancreatic beta cells, causing diabetes (Biochem Biophys Res Commun 363:140:2007). It is also revealed that pancreatic cells of diabetes patients are more susceptible to ER stress than are those of normal patients (Diabetologia 50:2486, 2007). These results imply that ER stress may be involved in the onset of insulin resistance and diabetes. Although the pathway of ER stress still remains unidentified, the UPR is known to start from PERK, IRE1 and ATF6, followed by various proteins including CHOP, XBP1, ATF4, GADD34, and eIF2a. Most of these ER stress-responsive proteins are transcription factors, and thus the target proteins transcribed by the factors arouse scientists' interest. Recent studies have reported that these ER stress-responsive proteins are involved in a variety of differentiation processes of adipocytes.

The X-box binding protein 1 (XBP-1) is a transcription factor containing a bZIP (basic leucine zipper) domain, which is upregulated as part of the ER stress response. Two forms of XBP1 have been identified: a spliced form, XBP1(S), and an unspliced form, XBP1(U). Splicing of the XBP1 mRNA by ER stress-induced active IRE1α results in the removal of a 26-base intron from XBP1(U) mRNA, creating a translational frameshift which leads to the active form XBP1(S) (Nature 415:92, 2002). The spliced protein product XBP1 is known to act as a potent transcriptional upregulator of many target genes in the unfolded protein response (UPR), an adaptive endoplasmic reticulum signaling pathway that allows cells to survive the accumulation of unfolded proteins in the endoplasmic reticulum lumen. Identified to have other various functions in addition to UPR, XBP1 is arising as an attractive target. As concerns diabetes and lipid metabolism, the XBP1 protein plays an important role in the regulation of insulin resistance (Science 306:457, 2004) and increases the synthesis of phosphatidylcholine, a main component of the cell membrane, to induce the expansion of the ER (Journal of Cell Biology 167:35, 2004; J Biol Chem 282:7024, 2007). Clinically, the activation of the XBP1 protein is closely related with the etiology of non-alcoholic fatty liver diseases (Gastroenterology Epub ahead of print, 2007).

U.S. Patent Publication No. 2006/0073213 discloses that ER stress including the expression of XBP1(S) is induced in the liver of obese mice and that XBP1(S) reduces ER stress to prevent the onset of diabetes, adding that obesity, hyperglycemia, insulin resistance or type II diabetes can be treated using ER stress reducers such as PBA (4-Phenyl-Butyric Acid), TLTDCA (Tauroursodeoxycholic Acid), and TMAO (Trimethylamine N-Oxide). Nowhere is the action or mechanism of the XBP1(S) protein on differentiation into adipocytes delineated in this application.

As described above, although a relationship between ER stress and obesity/diabetes and methods of treating obesity and diabetes using the relationship are disclosed, the interconnection or relation of the XBP1 protein with differentiation into adipocytes, a main cause of obesity, remains to be shown.

### DISCLOSURE

### Technical Problem

Leading to the present invention, intensive and thorough research into the effect of XBP1 on differentiation from pre-adipocytes, precursor cells or stem cells to adipocytes, conducted by the present invention, resulted in the finding that the expression level of XBP1(S) increases in proportion to differentiation into adipocytes while when XBP1(S) expression is repressed, the differentiation is also repressed, indicating that XBP1(S) regulates differentiation into adipocytes.

It is therefore an object of the present invention to provide the use of XBP1 in differentiation into adipocytes.

It is another object of the present invention to provide a protein marker, indicative of an increase in differentiation from pre-adipocytes, adipocyte precursor cells or stem cells into adipocytes, comprising as an active ingredient an XBP1(S) {X box-binding protein 1, spliced form} having an amino acid sequence of SEQ ID NO. 1, 2 or 3.

It is a further object of the present invention to provide a promoter of adipocyte differentiation, comprising at least one selected from an XBP1(S) protein or a polypeptide fragment having physiological activity identical to that of the XBP1(S) protein.

It is still a further object of the present invention to provide a repressor of adipocyte differentiation, comprising an XBP1(S) expression inhibitor.

It is still another object of the present invention to provide a method for promoting adipocyte differentiation, comprising: introducing into pre-adipocytes, adipocyte precursor cells or stem cells a promoter of adipocyte differentiation comprising at least one selected from a group consisting of an XBP1(S) protein, a polypeptide fragment having physiological activity identical to that of the XBP1(S) protein, and an XBP1(S) expression inducer; measuring a rate of differentiation into adipocytes; and further introducing the promoter of adipocyte differentiation when the rate of differentiation is lower than a predetermined amount.

It is yet a further object of the present invention to provide a method for repressing adipocyte differentiation, comprising: introducing into pre-adipocytes, adipocyte precursor cells or stem cells a repressor of adipocyte differentiation comprising an XBP1(S) expression inhibitor; measuring a rate of differentiation into adipocytes; and further introducing the repressor when the rate of differentiation is higher than a predetermined amount.

It is yet another object of the present invention to provide an agent for screening a repressor of adipocyte differentiation, comprising an XBP1(S) gene.

It is yet still a further object of the present invention to provide an agent for screening a repressor of adipocyte differentiation, comprising at least one selected from an XBP1(S) protein and a polypeptide fragment having the same physiological activity as that of the XBP1(S) protein.

It is yet still another object of the present invention to provide a method for screening a repressor of adipocyte differentiation, comprising: bringing the agent for screening a repressor of adipocyte differentiation comprising an XBP1(S) gene into contact with a test material; and determining whether the test material promotes or represses XBP1(S) expression.

It is still yet a further object of the present invention to provide a method for screening a repressor of adipocyte differentiation, comprising: bringing into contact with a test material an agent for screening a repressor of adipocyte differentiation comprising at least one selected from among XBP1(S) proteins and polypeptide fragments having the same physiological activity as that of the XBP1(S) proteins; and determining whether the test material is promotive or repressive of the adipocyte differentiation inducing activity of the XBP1(S) proteins or the polypeptide fragments having the same physiological activity as that of the XBP1(S) proteins.

It is still yet another object of the present invention to provide a method for reducing rosiglitazone's side effect of causing obesity, comprising repressing XBP1(S) expression when rosiglitazone is administered in the treatment of diabetes.

It is another object of the present invention to provide a protein marker, indicative of an increase in differentiation from pre-adipocytes, adipocyte precursor cells or stem cells into adipocytes, comprising as an active ingredient an XBP1(U) {X box-binding protein 1, unspliced form} having an amino acid sequence of SEQ ID NO. 4, 5 or 6.

### Technical Solution

In accordance with an aspect thereof, the present invention pertains to the regulation of differentiation into adipocytes. More particularly, the present invention pertains to a protein marker for indicating adipogenesis through the differentiation of preadipocytes, adipocyte precursor cells or stem cells into adipocytes, comprising as an active ingredient the XBP1(S) {X box-binding protein 1, spliced form} having an amino acid sequence selected from among SEQ ID NOS. 1 to 3, and the uses thereof in developing a promoter of adipocyte differentiation and a method for promoting adipocyte differentiation, in developing a repressor of adipocyte differentiation and a method for repressing adipocyte differentiation, an agent and a method for screening a repressor of adipocyte differentiation, and a method for reducing rosiglitazone's side effect of causing obesity.

Also, the present invention pertains to a protein marker, capable of indicating an increase in differentiation from pre-adipocytes, adipocyte precursor cells or stem cells into adipocytes, comprising as an active ingredient an XBP1(U) {X box-binding protein 1, unspliced form} having an amino acid sequence of SEQ ID NO. 4, 5 or 6.

### Advantageous Effects

Having the activity of regulating differentiation from pre-adipocytes, adipocyte precursor cells or stem cells into adipocytes, as described above, the XBP1(S) proteins of the present invention can be used for providing an adipogenic marker, a promoter of adipocyte differentiation, a repressor of adipocyte differentiation, a method for promoting adipocyte differentiation, a method for repressing adipocyte differentiation, a material for screening a repressor of adipocyte differentiation and a screening method using the same, and a method for reducing rosiglitazone's side effect of causing obesity.

### DESCRIPTION OF DRAWINGS

FIG. 1 is of optical photographs showing the morphological change of pre-adipocytes into adipocytes with differentiation time.

FIG. 2 is of histograms showing mRNA expression patterns of the adipogenic markers PPARγ2 (left) and ap2 (right) during differentiation from human pre-adipocytes into adipocytes.

FIG. 3 is of histograms showing expression patterns of XBP1(S) mRNA (left) and XBP1(U) mRNA (right) during differentiation from human pre-adipocytes into adipocytes.

FIG. 4 is of photographs showing expression patterns of XBP1(S) protein and XBP1(U) protein during differentiation from human pre-adipocytes into adipocytes, with APDH serving as a control for quantitative analysis

FIG. 5 is a histogram showing mRNA expression patterns of the adipogenic marker PPARγ2 during differentiation from human pre-adipocytes into adipocytes.

FIG. 6 is of histograms showing expression patterns of XBP1(S) mRNA (left) and XBP1(U) mRNA (right) during the differentiation of rat bone marrow stem cells into adipocytes.

FIG. 7 is a photograph showing expression patterns of XBP1(S) protein and XBP1(U) protein during the differentiation of rat bone marrow stem cells into adipocytes, with GAPDH serving as a control for quantitative analysis.

FIG. 8 is of optical photographs showing a morphological change of 3T3-L1 adipocyte precursor cells into adipocytes with induction time.

FIG. 9 is of histograms showing mRNA expression patterns of the adipogenic markers PPARγ2 (left) and ap2 (right) during differentiation of 3T3-L1 adipocyte precursor cells into adipocytes.

FIG. 10 is of histograms showing expression patterns of XBP1(S) mRNA (left) and XBP1(U) mRNA (right) during differentiation of 3T3-L1 adipocyte precursor cells into adipocytes.

FIG. 11 is of photographs showing expression patterns of XBP1(S) protein and XBP1(U) protein during the differentiation of 3T3-L1 adipocyte precursor cells into adipocytes, with GAPDH serving as a control for quantitative analysis.

FIG. 12 is of optical photographs showing morphological changes of the XBP1(S)-knock-down 3T3-L1 precursor cells (SFG-xbp1i), together with the controls (SFG-neo and 3T3-L1), differentiating into adipocytes with induction time.

FIG. 13 is of histograms showing expression patterns of XBP1(S) mRNA (left) and PPARγ2 mRNA (right) during differentiation of XBP1(S)-knock-down 3T3-L1 precursor cells into adipocytes.

FIG. 14 is a histogram showing expression patterns of markers and genes related to adipocyte differentiation and lipogenesis on Day 8 after differentiation from the XBP1(S)-knock-down 3T3-L1 precursor cells to adipocytes.

FIG. 15 is a histogram showing the regulation of XBP1(S) on PPARγ2 transcription through a comparison of luciferase activity between the 3T3-L1 cells cotransfected with the pGL3-PPARγ2 recombinant vector and the pcDNA3.1-XBP1(S) recombinant vector and the 3T3-L1 precursor cells cotransfected with the pGL3-PPARγ2 recombinant vector and the pcDNA3.1 vector.

FIG. 16 is a graph showing expression patterns of XBP1(S) mRNA in obese rats according to weight gain.

FIG. 17 is of histograms showing changes in XBP1(S) mRNA expression levels of HFD-fed mice with HFD-feeding time period, and a comparison in XBP1(S) mRNA expression level between ND- and HFD-fed mice on Week 8.

FIG. 18 is of optical photographs showing how rosiglitazone-induced increase of PPARγ2 mRNA expression level affects differentiation of XBP1(S)-knock-down 3T3-L1 precursor cells (SFG-xbp1i) into adipocytes in terms of morphology.

FIG. 19 is a histogram showing the effect of rosiglitazone on the expression of PPARγ2 mRNA upon differentiation of XBP1(S)-knock-down 3T3-L1 precursor cells (SFG-xbp1i) into adipocytes.

FIG. 20 is of photographs showing the effect of rosiglitazone on the expression of XBP1(S) upon differentiation of XBP1(S)-knock-down 3T3-L1 precursor cells (SFG-xbp1i) into adipocytes, with GAPDH serving as a control for quantitative analysis.

### BEST MODE

The XBP1 protein, as described above, belonging to the bZIP family, was first found to associate with the X box site in the promoter region of major histocompatibility complex (MHC) class II (Genes Cell 8:189, 2003). XBP1 mRNA is processed into an active form through the splicing mechanism mediated by ER stress-induced active IRE1 (inositol-requiring enzyme 1) when abnormal proteins accumulated in the ER are sensed as ER stress, thus producing a 26-base spliced mRNA from which the functionally active transcription factor XBP1(S) (X box-binding protein 1, spliced form) can be translated. On the other hand, blocking the transcription of a target gene when cells are recovering from endoplasmic reticulum stress, the unspliced XBP1(U) (X box-binding protein 1, unspliced form) protein encoded by XBP1 pre-mRNA acts as a negative feedback regulator on XBP-1(S) (J Cell Biol 172:565, 2006).

So long as it properly functions as a transcription factor, XBP1(S) may have any amino acid sequence in accordance with the present invention, but preferably is selected from among amino acid sequences of SEQ ID NOS. 1 to 3. The XBP1(S) protein useful in the present invention may be expressed when inducing differentiation of human adipocyte precursor cells into adipocytes (SEQ ID NO. 1, available as Accession No. BAB82982 on the NCBI database), from rat bone marrow stem cells into adipocytes (SEQ ID NO. 2, determined from the XBP1(U) mRNA of Accession No. NM_{—}001004210 on the NCBI database) or of mouse 3T3-L1 adipocyte precursor cells into adipocytes (SEQ ID NO. 3, available as Accession No. AAH16079 on the NCBI database).

Examples of the amino acid sequences of XBP1(U) useful in the present invention include SEQ ID NOS. 4 to 6, but are not limited thereto. The amino acid sequences of SEQ ID NO. 4 (available as Accession No. BAB82981 on the NCBI database), SEQ ID NO. 5 (available as Accession No. NP_{—}001004210 on the NCBI database) and SEQ ID NO. 6 (available as Accession No. NP_{—}038870 on the NCBI database) account for the XBP1(U) proteins expressed when introducing differentiation of adipocytes from human adipocyte precursor cells, rat bone marrow stem cells and mouse 3T3-L1 adipocyte precursor cells, respectively.

In the present invention, no particular limitations are imparted to pre-adipocytes, adipocyte precursor cells, and stem cells which will differentiate into adipocytes, but they are preferably derived from mammals including humans, rats and mice. The stem cells are preferably derived from rats and more preferably from the rat bone marrow. The adipocyte precursor cells are preferably derived from mice and are more preferably mouse 3T3-L1 precursor cells.

In accordance with an embodiment thereof, the present invention provides a protein marker for indicating adipogenesis through differentiation from pre-adipocytes, adipocyte precursor cells or stem cells into adipocytes, comprising as an active ingredient an XBP1(S) protein (X box-binding protein 1, spliced form) having an amino acid sequence of SEQ ID NO. 1, 2 or 3.

When adipocytes are differentiated from pre-adipocytes, precursor cells or stem cells, the XBP1(S) proteins of the present invention and the mRNAs thereof increase in expression level in proportion to the progress of adipogenesis. Also, the expression level of the XBP1(S) protein is proportional to that of the XBP1(U) when adipogenesis is induced through differentiation from pre-adipocytes, adipocyte precursor cells or stem cells to adipocytes. In addition, the XBP1(S) proteins of the present invention function to promote adipocyte differentiation when increasing in expression level and vice versa. That is, a decreased expression level of the XBP1(S) induces repression of adipocyte differentiation.

Expressed when adipocytes are induced to differentiate from pre-adipocytes, adipocyte precursor cells or stem cells, the XBP1(S) protein functions as a transcription factor for regulating the differentiation of pre-adipocytes, adipocyte precursor cells or stem cells into adipocytes and thus can be used as an adipogenic marker for the differentiation of adipocytes from pre-adipocytes, adipocyte precursor cells or stem cells. Further, in cases where adipogenesis increases when inducing the differentiation of pre-adipocytes, adipocyte precursor cells or stem cells into adipocytes, the expression level of the XBP1(S) mRNA is increased in proportion to that of the adipogenic marker PPARγ2 protein or ap2 protein. Accordingly, the XBP1(S) protein of the present invention can be used, alone or in combination with PPARγ2 or ap2, as an adipogenic maker.

In accordance with another embodiment thereof, the present invention provides a promoter of adipocyte differentiation comprising at least one selected from the XBP1(S) proteins of the present invention and polypeptide fragments identical in physiological function thereto.

With the increase in expression level, the XBP1(S) proteins of the present invention function to promote adipocyte differentiation. Hence, the XBP1(S) proteins or functionally identical polypeptide fragments can promote adipocyte differentiation, causing the hyperplasia of mature adipocytes.

The promoter of adipocyte differentiation may be useful in curing a significant number of patients suffering from the states or diseases caused by the general or partial absence of adipose tissues. Additionally, the adipocyte differentiation promoter may be used to induce the differentiation of stem cells into mature adipocytes, thus producing transplantable adipose tissues.

In accordance with a further embodiment thereof, the present invention provides an inhibitor of adipocyte differentiation comprising an agent for inhibiting the expression of XBP1(S).

Functioning by inhibiting the expression of XBP1(S) when pre-adipocytes, adipocyte precursor cells or stem cells are induced to differentiate into adipocytes, the inhibitor of adipocyte differentiation of the present invention can repress or block the hyperplasia of mature adipocytes.

The agent for inhibiting the expression of XBP1(S) according to the present invention may be in the form of nucleic acids, proteins, other extracts or naturally occurring materials, and is preferably siRNA (small interfering RNA) complementary to XBP1(S) mRNA. In accordance with the present invention, the siRNA is derived from shRNA (small hairpin RNA).

There are two methods of repressing the expression of XBP1(S): knock-out and knock-down. The latter, based on RNA interference (hereinafter referred to as “RNAi”), is more extensively used because it takes advantage of base sequence specificity and is simpler and more rapid in inducing the repression of gene expression than is the former. RNAi is an miRNA(micro RNA)- or siRNA(small interfering RNA)-induced mechanism of repressing gene expression by degrading an RNA molecule of interest or by interfering with the transcription of a gene of interest.

An siRNA, a 20˜25 nt long double-stranded RNA (hereinafter referred to as “dsRNA”) molecule, is associated with protein components to form RISC (RNA-induced silencing complex) which binds in turn complementarily to a target mRNA so as to cleave the target mRNA. In vivo, the cleavage of dsRNA or shRNA (small hairpin RNA) into siRNA is catalyzed by a dicer. Also, siRNA may be directly synthesized ex vivo. It may be introduced into cells using various transfection techniques, for example, with the aid of an siRNA expression vector designed to express siRNA within cells.

In accordance with still a further embodiment thereof, the present invention provides a method for promoting adipocyte differentiation, comprising: introducing into pre-adipocytes, adipocyte precursor cells or stem cells a promoter of adipocyte differentiation comprising at least one selected from a group consisting of XBP1(S) proteins, polypeptide fragments having physiological activity identical to that of the XBP1(S) proteins, and XBP1(S) expression inducers; measuring a rate of differentiation into adipocytes; and further introducing the promoter of adipocyte differentiation when the rate of differentiation is lower than a predetermined amount.

Also, the present invention provides a method for repressing adipocyte differentiation, comprising: introducing into pre-adipocytes, adipocyte precursor cells or stem cells a repressor of adipocyte differentiation comprising an XBP1(S) expression inhibitor; measuring a rate of differentiation into adipocytes; and further introducing the repressor when the rate of differentiation is higher than a predetermined amount.

In accordance with the present invention, the XBP1(S) expression differentiation inducer or inhibitor may be in the form of nucleic acids, proteins or other extracts or naturally occurring materials. Particularly, the XBP1(S) expression inhibitor preferably comprises siRNA (small interfering RNA) complementary to XBP1(S) mRNA.

The XBP1(S) protein of the present invention functions to promote differentiation into adipocytes when its expression level increases and to repress differentiation into adipocytes when its expression level decreases. If a rate of the differentiation of pre-adipocytes, adipocyte precursor cells or stem cells into adipocytes, when measured, is higher or lower than a predetermined amount, the promoter or repressor of adipocyte differentiation is introduced at a desired level within cells to regulate the rate of differentiation into adipocytes.

The introduction of the promoter or repressor of adipocyte differentiation into cells may be carried out in various manners such as administering into mammals a drug formulated with the promoter or repressor of adipocyte differentiation and using a genetic engineering vector system.

In the present invention, the measuring of a rate of differentiation into adipocytes may comprise determining differentiation into adipocytes at a morphological level, an XBP1(S) mRNA level or an XBP1(S) protein level. Preferably, the differentiation rate can be measured by an Oil-Red O staining method for morphological analysis, real-time PCR (Polymerase chain reaction) for XBP1(S) mRNA analysis and immunoblotting for XBP1(S) protein analysis, but the present invention is not limited thereto.

In accordance with still another embodiment thereof, the present invention provides an agent for screening a repressor of adipocyte differentiation, comprising an XBP1(S) gene. Also, the present invention provides an agent for screening a repressor of adipocyte differentiation, comprising at least one selected from XBP1(S) proteins and polypeptide fragments having the same physiological activity as that of XBP1(S) proteins.

The XBP1(S) gene, useful as an agent for screening a repressor of adipocyte differentiation in the present invention, preferably comprises an mRNA or cDNA encoding an amino acid sequence selected from among SEQ ID NOS. 1 to 3.

The agent for screening a repressor of adipocyte differentiation in accordance with the present invention will find an application in developing a drug for the prevention or treatment of obesity.

In accordance with yet another embodiment thereof, the present invention provides a method for screening a repressor of adipocyte differentiation, comprising: bringing an agent for screening a repressor of adipocyte differentiation into contact with a test material; and determining whether the test material is promotive or repressive of XBP1(S) expression.

Also, the present invention provides a method for screening a repressor of adipocyte differentiation, comprising: bringing into contact with a test material an agent for screening a repressor of adipocyte differentiation comprising at least one selected from among XBP1(S) proteins and polypeptide fragments having the same physiological activity as that of the XBP1(S) proteins; and determining whether the test material is promotive or repressive of the adipocyte differentiation inducing activity of the XBP1(S) proteins or the polypeptide fragments having the same physiological activity as that of the XBP1(S) proteins.

In the screening method of the present invention, a reaction between the XBP1(S) gene and the test material can be detected using a typical technique used for determining DNA-DNA, DNA-RNA, DNA-protein and DNA-compound reactions. For example, useful is in vitro hybridization between the gene and the test material, Northern analysis after reaction between cells and the test material, gene expression rate analysis through quantitative PCR or quantitative real-time PCR, or reporter expression analysis in which a reporter gene linked to the XBP1(S) gene is introduced into cells and allowed to react with the test material and a reporter expression rate is measured. In this context, the screening agent of the present invention may comprise distilled water or buffer for stabilizing a nucleic acid structure in addition to the XBP1(S) gene.

In the screening method of the present invention, a reaction between the XBP1(S) proteins or the polypeptide fragments having the same physiological activity as that of XBP1(S) and the test material can be detected using a typical technique for determining a reaction between protein-protein or protein-compound. Useful is, for example, activity measurement after reaction between the XBP1(S) gene or XBP1(S) protein and the test material, yeast two-hybrid, screening of phage-displayed peptide clones binding to XBP1(S), HTS (high throughput screening) utilizing a natural material or chemical library, drug hit HTS, cell-based screening, or screening using a DNA array. In this context, the screening agent of the present invention may comprise a buffer or solvent for stabilizing protein structures or physiological activity in addition to the XBP1(S) proteins or the polypeptide fragments having the same physiological activity as that of XBP1(S). For an in vivo test, the screening agent may further comprise a cell expressing the protein or a cell carrying a plasmid expressing the protein in the presence of a promoter.

The test material useful in the screening method of the present invention may be a nucleic acid, a protein or an extract or naturally occurring material which is a putative repressor of adipocyte differentiation or a randomly selected nucleic acid, protein or another extract or naturally occurring material.

When identified to show inhibitory activity against XBP1(S) expression or function in the screening method of the present invention, the test material can be a candidate for a repressor of adipocyte differentiation. On the other hand, when identified to promote XBP1(S) expression or function in the screening method of the present invention, the test material may be used to develop an antagonist thereto which may be a candidate for a repressor of adipocyte differentiation.

Of course, the candidates for a repressor of adipocyte differentiation will be treated as leading compounds for developing a repressor of adipocyte differentiation. When it comes to development, such leading compounds are typically structurally modified and optimized so as to elicit inhibitory effects on the functioning of XBP1(S).

Showing partial or complete inhibitory activity against XBP1(S) genes or proteins, the materials thus obtained can be used for the treatment of diseases related to the hyperfunction of the XBP1(S) gene or protein leading to the induction of adipocyte differentiation, such as obesity, type 2 diabetes, etc.

In accordance with yet a further embodiment thereof, the present invention provides a method for reducing rosiglitazone's side effect of causing obesity, comprising repressing XBP1(S) expression when rosiglitazone is used in the treatment of diabetes.

Rosiglitazone is an anti-diabetic drug. It acts through the activation of PPARγ2 and is clinically used to treat diabetes, but with the side effect of causing obesity.

According to the present invention, when inducing the differentiation of adipocytes from adipocyte precursor cells in which XBP1(S) expression is suppressed, the addition of rosiglitazone increases the expression of PPARγ2 mRNA as well as XBP1(S) mRNA. In spite of the treatment of diabetes, rosiglitazone induces an increase in XBP1(S) expression level, so that the XBP1(S) thus increased binds to the promoter of PPARγ2 to activate the transcription of PPARγ2, which in turn promotes adipocyte differentiation with the side effect of causing obesity.

Even in the presence of rosiglitazone, the XBP1(S) expression inhibitor according to the present invention can repress XBP1(S) expression, thus reducing rosiglitazone's side effect of causing obesity. The XBP1(S) expression inhibitor still effective even in the presence of rosiglitazone may be selected from the candidates for a repressor of adipocyte differentiation screened by the screening method and may be in the form of nucleic acids, proteins and other extracts or naturally occurring materials. The repression of XBP1(S) expression in the treatment of diabetes with rosiglitazone may be achieved by administering an XBP1(S) expression inhibitor prior to XBP1(S) expression subsequently to or concurrently with rosiglitazone.

In accordance with still yet a further embodiment thereof, the present invention provides a protein marker useful as an adipogenic indicator predictive of differentiation of pre-adipocytes, adipocyte precursor cells or stem cells into adipocytes, comprising as an active ingredient an XBP1(U) protein {X box-binding protein 1, unspliced form} having an amino acid sequence selected from among SEQ ID NOS. 4 to 6.

XBP(U) proteins and their mRNAs are expressed prior to differentiation into adipocytes and the expression level thereof is in proportion to differentiation into adipocytes. In addition, the XBP(U) protein of the present invention is expressed prior to the expression of XBP1(S) and the expression level of XBP(U) is proportional to that of XBP1(S).

Therefore, the expression level of XBP(U) upon differentiation of pre-adipocytes, adipocyte precursor cells or stem cells into adipocytes can be predictive of whether adipocyte differentiation will increase or decrease.

### MODE FOR INVENTION

A better understanding of the present invention may be obtained through the following examples which are set forth to illustrate, but are not to be construed as limiting the present invention.

### Assays

### Assay Method 1: Morphological Analysis of Adipocytes (Oil-Red O Staining)

For the morphological determination of differentiation from pre-adipocytes to adipocytes, Oil-red O staining was performed. Cells which had been subjected to differentiation were washed with PBS (phosphate buffered saline) and fixed with 10% formalin. After fixation for 10 min, the cells were cleansed with distilled water and stained for 10 min with a 0.3% Oil-red O solution. Subsequently, the cells were washed with 60% isopropanol and distilled water to remove the staining solution and then mounted on slides.

Assay Method 2: mRNA Expression Analysis (Real-Time PCR)

From the cells subjected to differentiation, total RNA was isolated by chloroform extraction and ethanol precipitation (TRIzol Reagent, Invitrogen, Carlsbad, Calif.). Thereafter, the total RNA was used to synthesize cDNA by RT-PCR (M-MLV reverse transcriptase, Promega, MADISON, Wis.). Real-time PCR was performed using a fluorescent probe of SYBR Green (SYBR Green QPCR Master Mix (2×), TAKARA, JAPAN), with cDNA serving as a template. Relative mRNA expression levels were calculated using a comparative Ct method.

### Assay Method 3: Qualitative and Quantitative Analysis of Protein (Immunoblot)

The cells which had been subjected to differentiation were treated with RIPA cell lysis buffer (20 mM Tris-HCl, pH 7.5, 0.1% SDS, 1% Triton X-100, 1% sodium deoxycholate, 150 mM NaCl, 1 mM EDTA, 1% NP-40, and proteinase inhibitor cocktail) to isolate proteins therefrom. In this regard, the cell culture was mixed with RIPA buffer, incubated for 30 min on ice and centrifuged for 30 min at 13,200×g. The supernatant was quantitatively assayed for protein using a Bradford method (Pierce, USA). 40 μg of the protein mixture was separated on 12% SDS-PAGE gel by electrophoresis and then transferred to a nitrocellulose membrane (Schleicher & Schell, USA). The membrane was blocked for 1 hr in 3% BSA (Bovine Serum Albumin), followed by immunoblotting against XBP1 with various antibodies including anti-XBP1 (1:200 dilution, Santa Cruz, USA), anti-GAPDH (1:1,000 dilution, Sigma), horseradish peroxidase-conjugated anti-rabbit and anti-mouse IgG (1:1,000 dilution, Santa Cruz). Immunoreactive bands were read with ECL (Amersham Pharmacia, USA).

### Assay Method 4: Measurement of Luciferase Activity

Luciferase activity was measured using the Dual Luciferase Assay Kit (Promega) in a luminometer.

### EXAMPLES

### Example 1

### Separation of Human Pre-Adipocytes, Adipocyte Differentiation and Expression Change

From an adult patient with abdominal obesity, adipose tissue was taken during suction lipectomy and aseptically carried to a laboratory. The adipose tissue was washed three times with PBS (phosphate buffered saline) containing anti-bacterial and anti-fungal agents and incubated for 60 min at 37° C. in PBS containing the same volumes of 1% bovine serum albumin and 0.1% collagenase type I (Collagenase type I, Invitrogen Corporation, Carlsbad, Calif.) as that of the tissue with shaking, so as to separate cells aseptically. Then, centrifugation was conducted for 10 min at 1000 rpm (Large Capacity table-top Centrifuge, Hanil Science, Incheon). The cell pellet thus formed was suspended in an alpha Minimum Essential Medium (alpha-MEM, Gibco BRL, Green Island, N.Y., USA), followed by filtration through a 40□ cell strainer (BD Bioscience, Two Oak Park, Bedford, Mass.) to remove remaining tissues. The filtrate was incubated at 37° C. in a 5% CO₂ incubator (CO₂ incubator, Model 3546, Forma scientific Inc., Marietta, Ohio). During incubation, the cells were observed for morphology under an inverted microscope (CK40, Olympus, Inc., Japan) everyday, and the culture medium was changed out with a fresh one every three days. When reaching about 80% confluency, the cells were separated with trypsin EDTA (Gibco/BRL), followed by three passages to afford pre-adipocytes.

For differentiation into adipocytes, the pre-adipocytes were seeded at a density of 50,000 cells/cm² and cultured to confluence (day 0). At this time, a differentiation inducing medium (induction medium) was added to allow differentiation for 20 days and the medium was changed out with a fresh one every four days. The differentiation inducing medium comprised 10 μg/ml insulin, 100 μg/m13-isobutyl-1-methylxanthine (IBMX), 50 μM indomethacin and 1 μM dexamethasone (Steraloids, Newport, R.I.) in MEM-a.

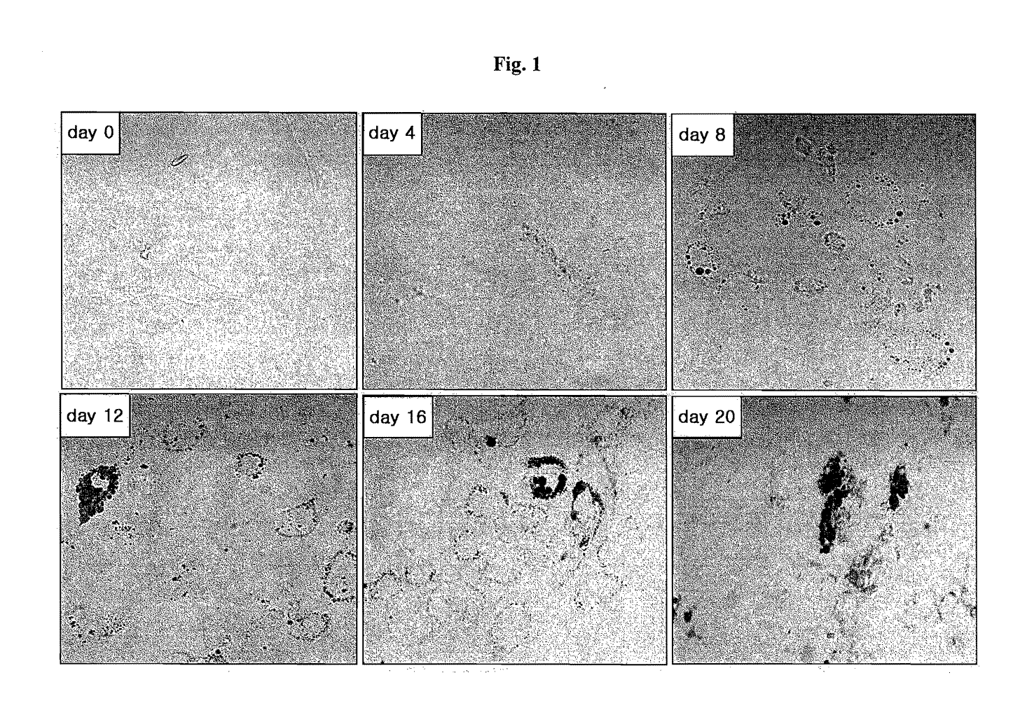

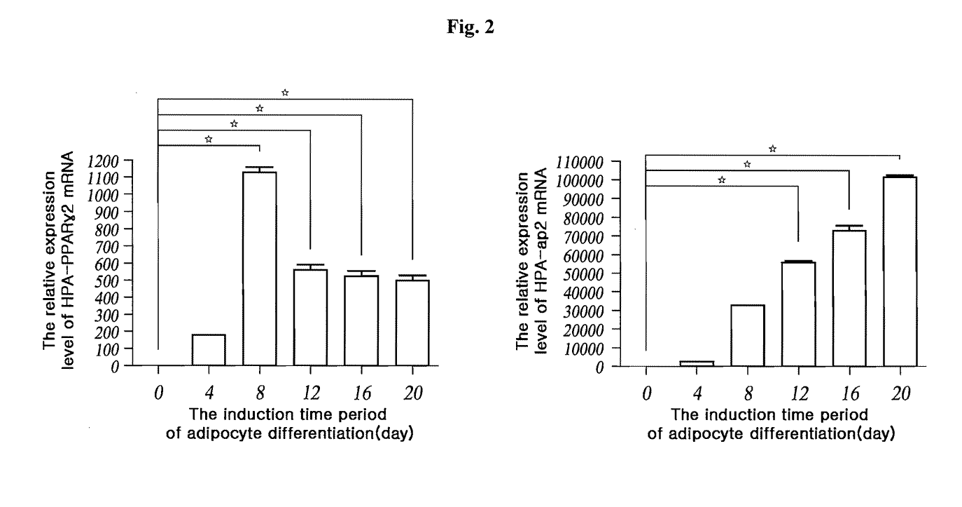

FIG. 1 shows the morphological change of pre-adipocytes into adipocytes with differentiation time. FIG. 2 shows mRNA expression patterns of the adipogenic markers PPARγ2 (left) and ap2 (right) during differentiation from human pre-adipocytes to adipocytes. As seen in the optical photographs of FIG. 1, adipocytes started to apparently appear on Day 8 after the induction of adipocyte differentiation, and their population increased until Day 20. In addition, as seen in FIG. 2, the expression level of PPARγ2 mRNA 1100-fold increased on Day 8 compared to that on Day 0 and remained in an increased pattern after Day 8. As for the relative expression level of ap2 mRNA, it sharply leaped at Day 8 and increased until Day 20 in a time-dependent pattern.

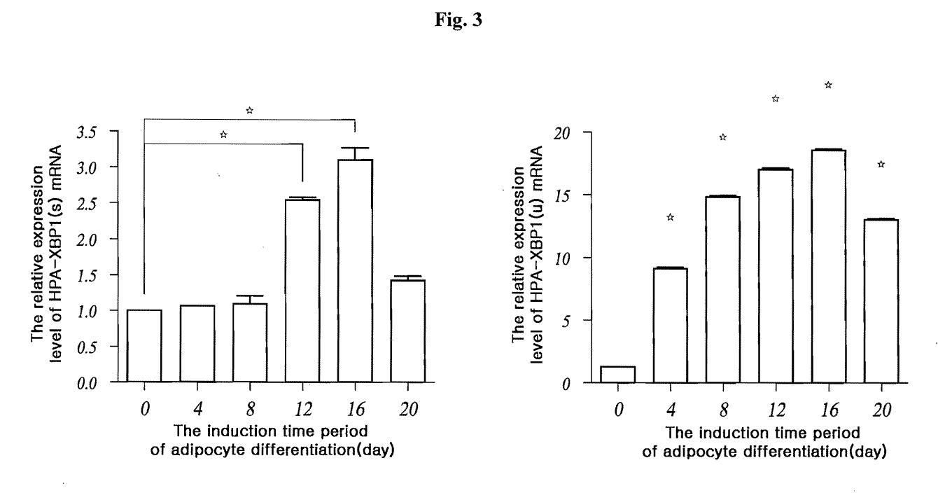

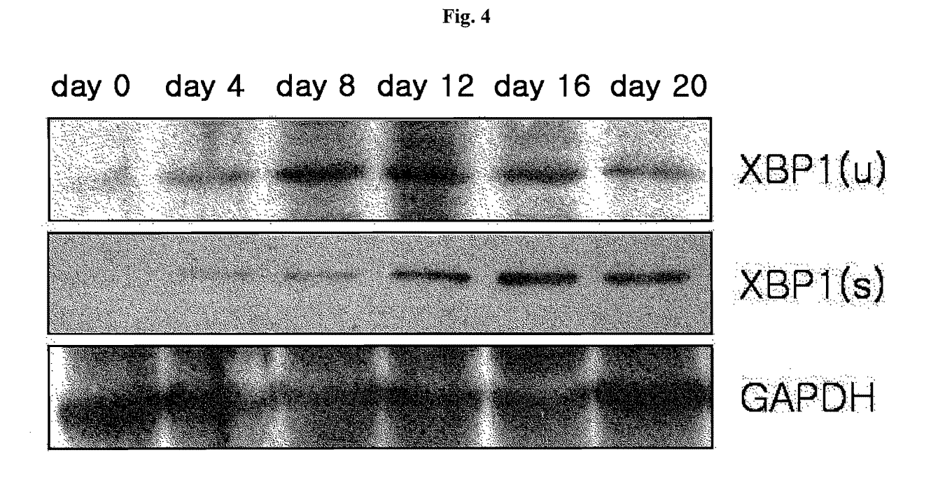

FIG. 3 shows expression patterns of XBP1(S) mRNA (left) and XBP1(U) mRNA (right) during differentiation from human pre-adipocytes to adipocytes. FIG. 4 shows expression patterns of XBP1(S) protein and XBP1(U) protein during differentiation from human pre-adipocytes to adipocytes. As is apparent from the graphs of FIG. 3, XBP1(S) mRNA started to significantly increase in expression level on Day 12 after the induction of adipocyte differentiation while the relative expression level of XBP1(U) mRNA increased in a time-dependent pattern starting with a significant leap on Day 4. Turning to FIG. 4, the expression level of XBP1(S) protein increased in a time-dependent pattern starting with a significant leap on Day 8. As concerns the expression level of XBP1(U) protein, it significantly increased on Day 4 after the induction of adipocyte differentiation with a peak on Day 12, and since then, showed a gradually decreasing pattern.

### Example 2

### Isolation of Rat Bone Marrow Stem Cells, Adipocyte Differentiation and Expression Change

After muscle was removed therefrom the femur (tibia) isolated from an adult rat was cut at its opposite ends. A syringe needle was inserted into the bone at the center of one end and an HF₂ medium was infused into the bond through the needle. The effluent flowing out of the bone was pooled and centrifuged (2500 rpm, 5 min). The supernatant was removed and the cell pellet was suspended and placed on Ficoll, followed by centrifugation (2500 rpm, 20 min). A band of the BMMC (Bone Marrow Mononuclear Cell) layer was taken. The cells thus obtained were cultured in a 20% Dulbecco's modified Eagle's medium (DMEM, Gibco BRL, Green Island, N.Y., USA). During the incubation, the cells were morphologically observed under an inverted microscope (CK40, Olympus, Inc., Japan), with the medium changed out with a fresh one every three days. When reaching about 80% confluence, the cells were separated with trypsin EDTA (Gibco/BRL) and subjected to three passages to afford rat bone marrow stem cells.

For differentiation into adipocytes, the rat bone marrow stem cells were seeded at a density of 50,000 cells/cm² and cultured to confluence (day 0). At this time, a differentiation inducing medium (induction medium) was added to allow differentiation for 20 days and changed out with a fresh one every four days. The differentiation inducing medium comprised 10 μM insulin, 0.5 mM 3-isobutyl-1-methylxanthine (IBMX), 0.2 mM indomethacin and 1 μM dexamethasone (Steraloids, Newport, R.I.) in DMEM.

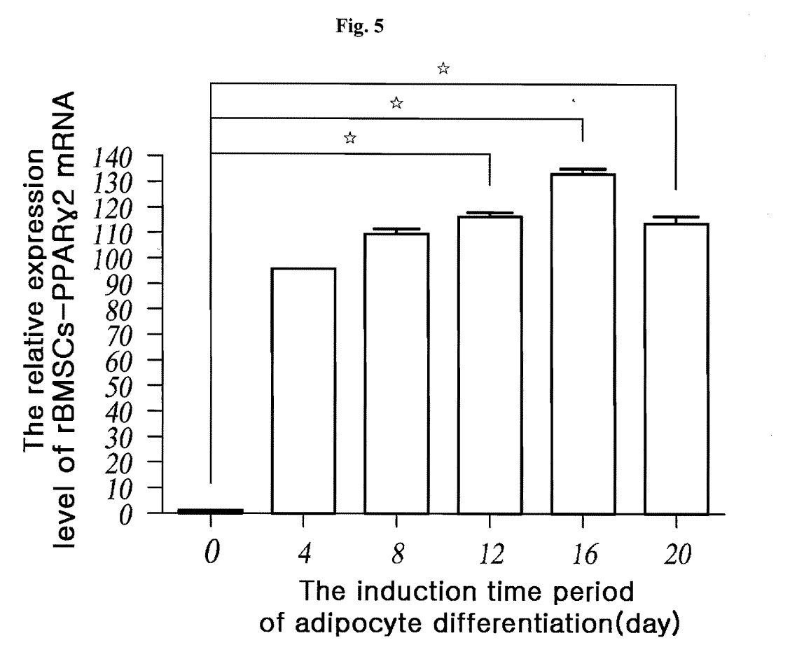

FIG. 5 shows mRNA expression patterns of the adipogenic marker PPARγ2 during differentiation from human pre-adipocytes to adipocytes. As seen in FIG. 5, the expression level of PPARγ2 mRNA started to increase on Day 4 after the induction of adipocyte differentiation.

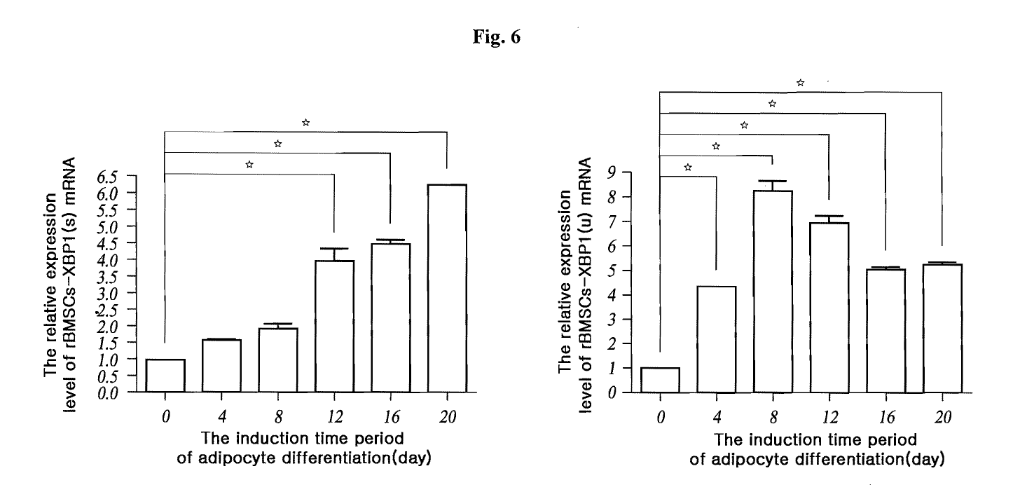

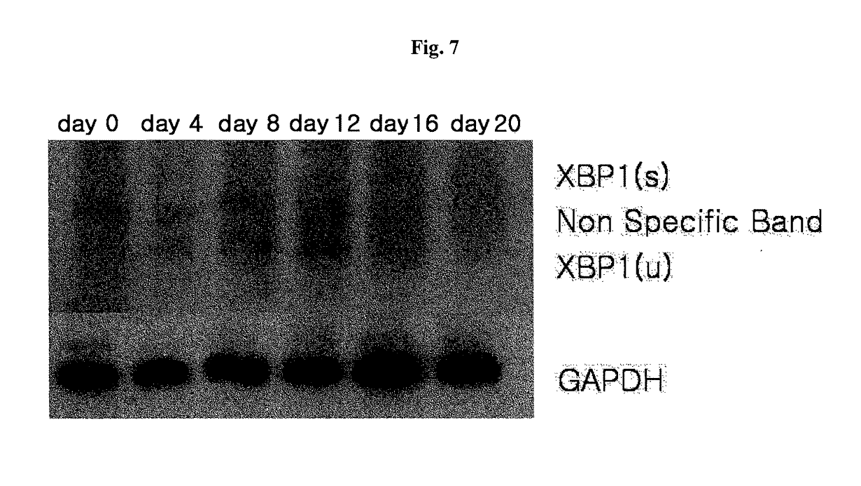

FIG. 6 shows expression patterns of XBP1(S) mRNA (left) and XBP1(U) mRNA (right) during differentiation from rat bone marrow stem cells to adipocytes. FIG. 7 shows expression patterns of XBP1(S) protein and XBP1(U) protein during differentiation from rat bone marrow stem cells to adipocytes. As is apparent from graphs of FIG. 6, the relative expression level of XBP1(S) mRNA was increased in a time-dependent pattern starting from Day 4 with a significant leap while the expression level of XBP1(U) mRNA significantly increased on Day 4 after the induction of adipocyte differentiation with a peak on Day 4, and since then, showed a gradually decreasing pattern. Turning to FIG. 7, the expression level of XBP1(S) protein increased in a time-dependent pattern starting with a significant leap on Day 8. As concerns the expression level of XBP1(U) protein, it significantly increased on Day 4 after the induction of adipocyte differentiation with a peak on Day 12, and since then, showed a gradually decreasing pattern.

### Example 3

### Culture of Mouse 3T3-L1 Adipocyte Precursor Cells, Adipocyte Differentiation and Expression Change

Mouse 3T3-L1 adipocyte precursor cells, purchased from American Type Culture Collection (ATCC, Manassas, Va., USA), were cultured in DMEM supplemented with 10% calf serum. For differentiation into adipocytes, the 3T3-L1 adipocyte precursor cells were seeded at a density of 50,000 cells/cm² and cultured to confluence and for an additional two days at confluence to reach a post-confluence (day 0). At this time, a differentiation inducing medium (induction medium) was added to allow differentiation for 8 days and it was changed out with a fresh one every other day. The differentiation inducing medium comprised 167 nM insulin, 0.5 mM 3-isobutyl-1-methylxanthine (IBMX) and 1 μM dexamethasone (Steraloids, Newport, R.I.) in 10% fetal bovine serum DMEM.

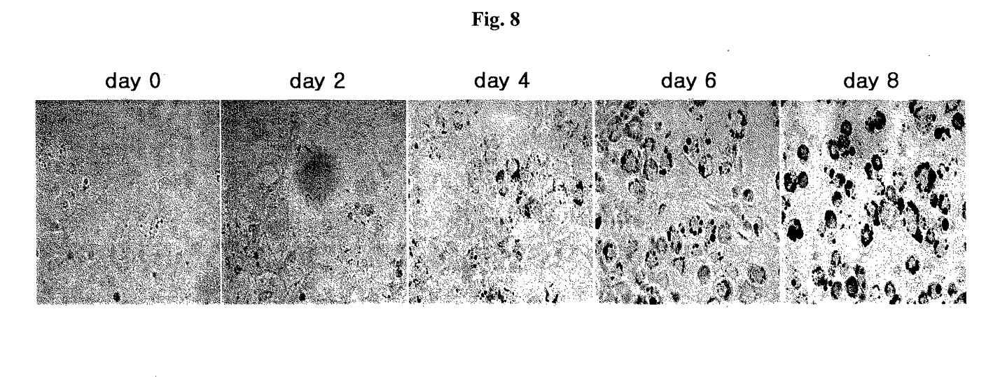

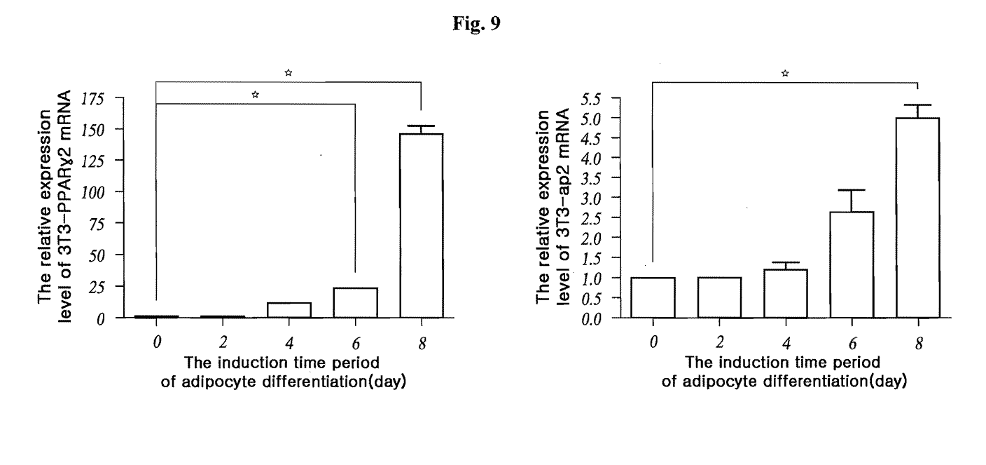

FIG. 8 shows a morphological change of 3T3-L1 adipocyte precursor cells into adipocytes with induction time. FIG. 9 shows mRNA expression patterns of the adipogenic markers PPARγ2 (left) and ap2 (right) during differentiation from 3T3-L1 adipocyte precursor cells to adipocytes. As seen in the optical photographs of FIG. 9, adipocytes started to apparently appear on Day 4 after the induction of adipocyte differentiation, and their population increased with induction time. In addition, as seen in FIG. 9, both the adipogenic markers PPARγ2 and ap2 had significantly increased mRNA expression levels in a time-dependent pattern, starting from Day 4 with a significant leap on Day 8. However, these cells were lower in the mRNA expression level of both PPARγ2 and ap2 than human pre-adipocytes during adipocyte differentiation. On Day 8, the mRNA expression level was 150-fold and 5-fold increased for PPARγ2 and ap2, respectively.

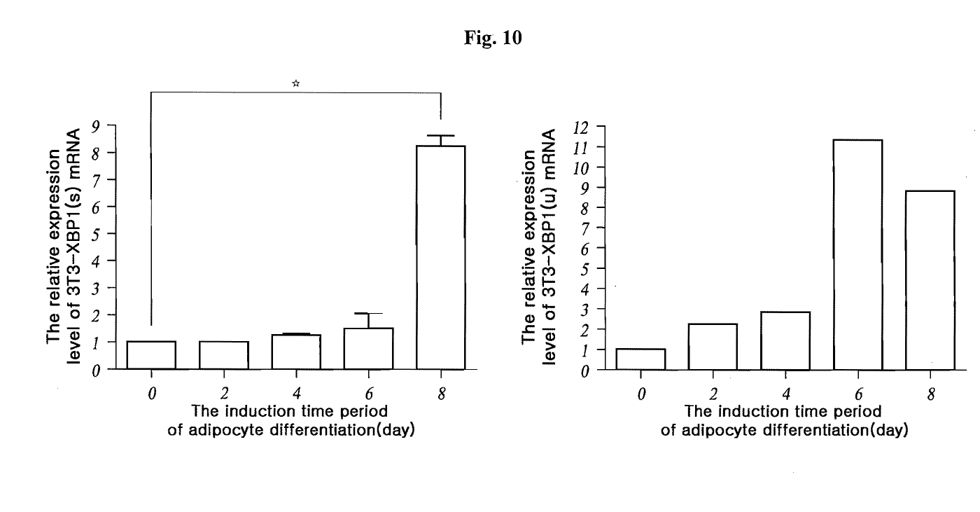

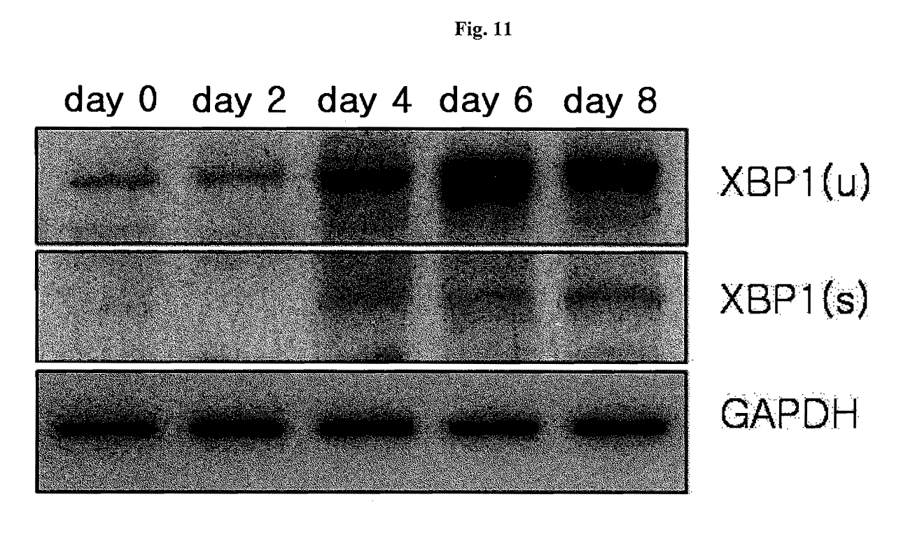

FIG. 10 shows expression patterns of XBP1(S) mRNA (left) and XBP1(U) mRNA (right) during differentiation from 3T3-L1 adipocyte precursor cells to adipocytes. FIG. 11 shows expression patterns of XBP1(S) protein and XBP1(U) protein during differentiation from 3T3-L1 adipocyte precursor cells to adipocytes. As is apparent from graphs of FIG. 10, the expression level of XBP1(S) mRNA started to significantly increase on Day 4 after the induction of adipocyte differentiation and showed a nonuple increase on Day 8 compared to that on Day 0 with an expression pattern similar to that of PPARγ2 and ap2 mRNA. On the other hand, the relative expression level of XBP1(U) mRNA significantly increased on Day 2 and peaked on Day 6, and since then, showed a gradually decreasing pattern. Turning to FIG. 11, the expression level of XBP1(S) protein significantly increased on Day 4 after the induction of adipocyte differentiation. As concerns the expression level of XBP1(U) protein, it started to increase from Day 0 with a sharp leap on Day 4.

The data obtained from Examples 1, 2 and 3 show that common to the differentiation of human pre-adipocytes, rat bone marrow stem cells and mouse 3T3-L1 adipocyte precursor cells into adipocytes follows an expression pattern similar to that between the transcription factor XBP1(S) protein and its mRNA and the adipogenic markers PPARγ2 and ap2, suggesting that XBP1(S) proteins can be used as an adipogenic marker and may play an important role in adipocyte differentiation. In addition, the XBP1(U) proteins and their mRNAs started to be expressed before adipocyte differentiation and increased in expression level in proportion to the extent of adipocyte differentiation. This pattern was found to be true of the expression of the XBP1(S) proteins and their mRNAs. Accordingly, the XBP1(U) proteins can be used as a marker predictive of adipocyte differentiation.

### Example 4

### Change in Adipocyte Differentiation with Repression of XBP1(S) Expression

In order to construct a retrovirus strain capable of expressing siRNA against XBP1(S) mRNA, SFG-shxbp1 and gag/pol and an env plasmid were transfected into human embryonic kidney (HEK) cell line 293T (Lipofectamine 2000 reagent, Invitrogen) to give a retroviral vector soup. The soup was infected into 3T3-L1 adipocyte precursor cells which were then incubated for 24 hrs with G418 to select infected cells which were repressed from expressing the XBP1(S) protein (SFG-xbp1i). This strain was found to repress the expression of XBP1(S) protein at 80% or higher. The cell lines infected with no retroviruses (3T3-L1) or only with a retrovirus incapable of expressing siRNA against XBP1(S) mRNA (SFG-neo) were used as controls.

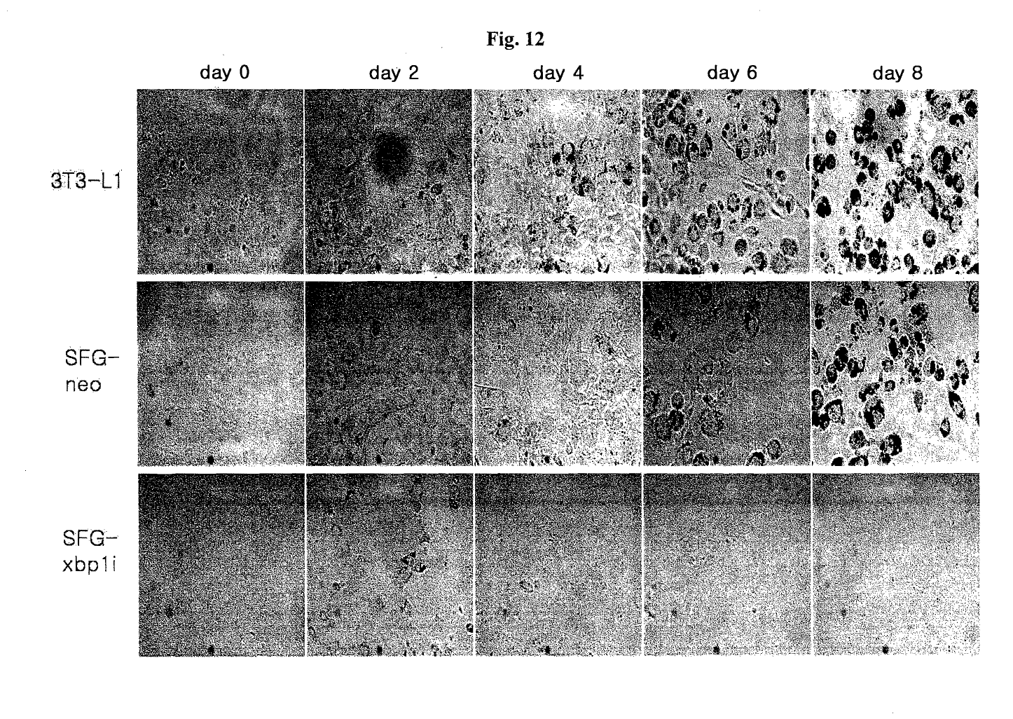

FIG. 12 shows the morphological change from the XBP1(S)-knock-down 3T3-L1 adipocyte precursor cells (SFG-xbp1i), together with the controls, to adipocytes with differentiation time. As seen in FIG. 12, both 3T3-L1 and SFG-neo stains underwent normal adipocyte differentiation whereas no adipocyte differentiation was found in the SFG-xbp1i cell strain in which the XPB1(S) was knock down.

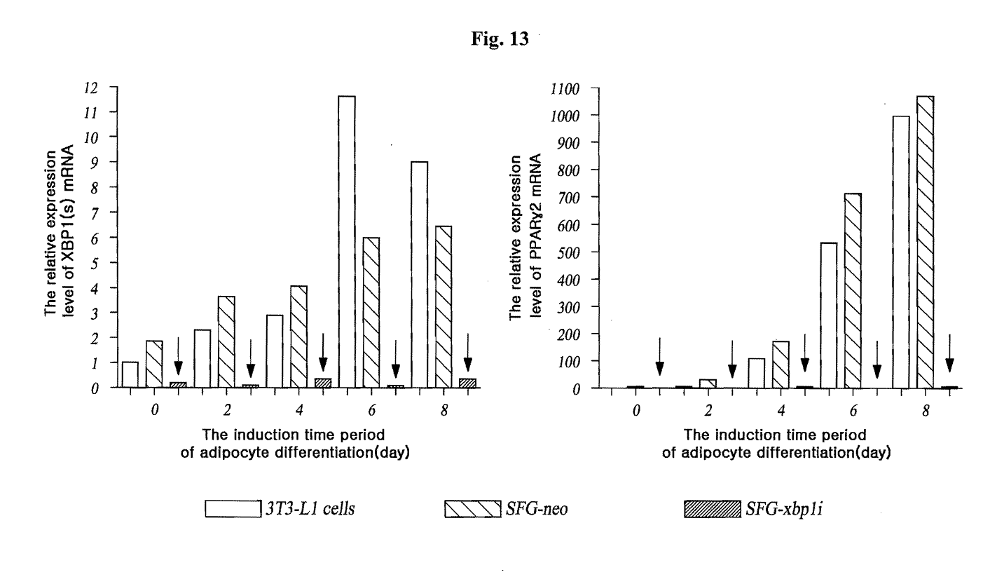

FIG. 13 shows expression patterns of XBP1(S) mRNA (left) and PPARγ2 mRN (right) during differentiation from the XBP1(S)-knock-down 3T3-L1 adipocyte precursor cells to adipocytes. As seen in FIG. 13, both 3T3-L1 and SFG-neo cells increased in the expression level of XBP1(S) mRNA and PPARγ2 mRNA with differentiation time. In contrast, XBP1(S) mRNA AND PPARγ2 mRNA were expressed only slightly in the SFG-xbp1i cells in which the expression of XPB1(S) protein was repressed.

Based on the observation that upon differentiation from XBP1(S) expression-repressed cells to adipocytes, almost no mRNAs of the adipogenic marker PPARγ2 were expressed with complete repression of adipocyte differentiation, Example 4 indicates that the XBP1(S) protein plays a pivotal role in regulating adipocyte differentiation.

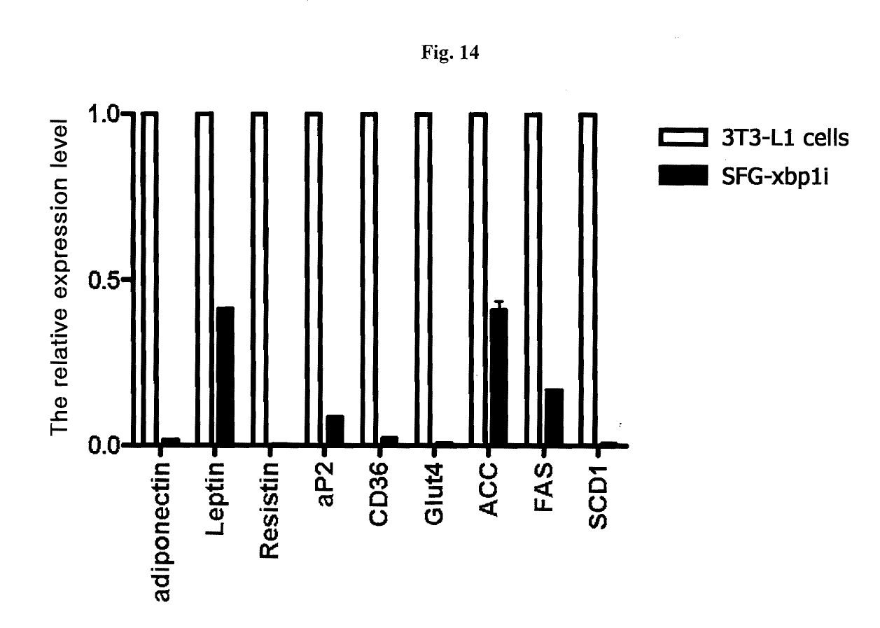

Turning to FIG. 14, it shows expression patterns of markers and genes related to adipocyte differentiation and lipogenesis on Day 8 after differentiation from the XBP1(S)-knock-down 3T3-L1 adipocyte precursor cells (SFG-xbp1i) to adipocytes. As seen in FIG. 14, SFG-xbp1i was observed to significantly decrease in the expression levels of all the markers including the adipogenic marker ap2; the cytokines known to be synthesized in adipocytes, adiponectin, leptin, and resistin; the fat trapping genes, CD36 and Glut4; and the lipogenic enzymes ACC (acetyl-CoA carboxylase), FAS (fatty acid synthase), SCD1 (stearoyl-CoA desaturase-1), compared to wild-type 3T3-L1 precursor cells.

### Example 5

### Transcriptional Regulation of XBP1(S) on PPARγ2

On the basis of the data that the expression of PPARγ2 mRNA is repressed upon the differentiation of XPB1(S)-knock-down 3T3-L1 precursor cells into adipocytes, an examination was made of how XBP1(S) regulated PPARγ2 transcription.

For this, the PPARγ2 promoter of wild-type 3T3-L1 precursor cells was cloned into a luciferase expression vector to construct a pGL3-PPARγ2 recombinant vector. Separately, wild-type 3T3-L1 precursor cells were seeded at a density of 1×10⁵ cells/well in 24-well plates, followed by cotransfection with the pGL3-PPARγ2 recombinant vector and the XBP1(S) expressing pcDNA3.1-XBP1(S) recombinant vector in the presence of Lipofectamin 2000 (Invitrogen, Carlsbad, Calif.). Instead of the pcDNA3.1-XBP1(S) recombinant vector, the pcDNA3.1 vector, which cannot express XBP1(S), was used as a control. 48 Hours after the cotransfection, the cells were measured for luciferase activity.

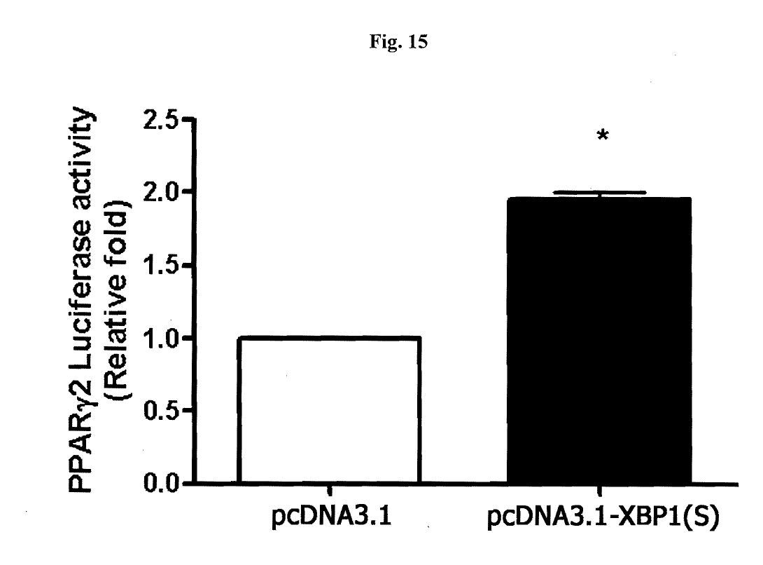

With reference to FIG. 15, luciferase activity in the 3T3-L1 cells cotransfected with the pGL3-PPARγ2 recombinant vector and the pcDNA3.1-XBP1(S) recombinant vector is compared with that in the 3T3-L1 precursor cells cotransfected with the pGL3-PPARγ2 recombinant vector and the pcDNA3.1 vector, showing the regulation of XBP1(S) on PPARγ2 transcription. As seen in FIG. 15, the cells transfected with the XBP1(S)-expressing pcDNA3.1-XBP1(S) recombinant vector were measured to have luciferase activity about twice as large as that of the cells transfected with the non-XBP1(S)-expressing pcDNA3.1 vector, indicating that XBP1(S) acts as a transcription factor by binding to the PPARγ2 promoter.

### Example 6

### In vivo Expression Change of XBP1(S) mRNA with Weight Gain of Obese Rat

An increase in differentiation from precursor cells to adipocytes results in the hyperplasia of adipocytes, which clinically accounts for obesity. The XBP1(S) proteins which were proven to promote adipocyte differentiation in vitro were examined to have the same activity in vivo. In this context, the adipose tissue of obese rats was analyzed for XBP1(S) mRNA expression level in relation to weight gain.

At 8, 16, 20, 33, 37 and 52 weeks after birth, OLETF rats (Otsuka Long-Evans Tokushima fatty rats, an animal model of type II diabetes with obesity), which undergo natural penetrance of diabetes and obesity, were weighed and assayed for XBP1(S) mRNA expression level in their visceral fat.

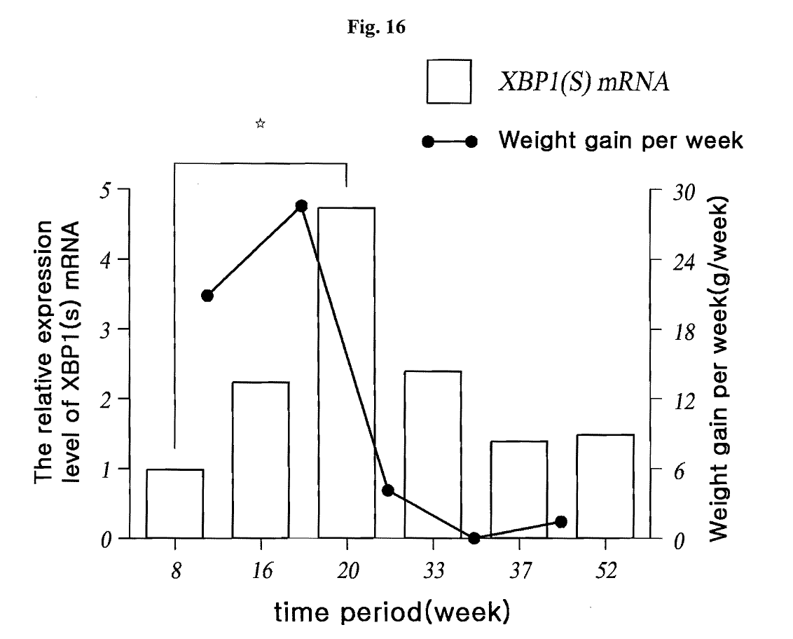

FIG. 16 is a graph showing expression patterns of XBP1(S) mRNA in obese rats as a function of weight gain. As seen in FIG. 16, the relative expression level of XBP1(S) mRNA in obese rats (histograms in FIG. 16) peaked at 20 weeks post-birth after birth and since then, declined. In addition, the weekly weight change of the obese rats (line plots in FIG. 16) was observed to have the same profile as that of the expression pattern of XBP1(S) mRNA, indicating that the expression level of XBP1(S) mRNA has a direct influence on weight.

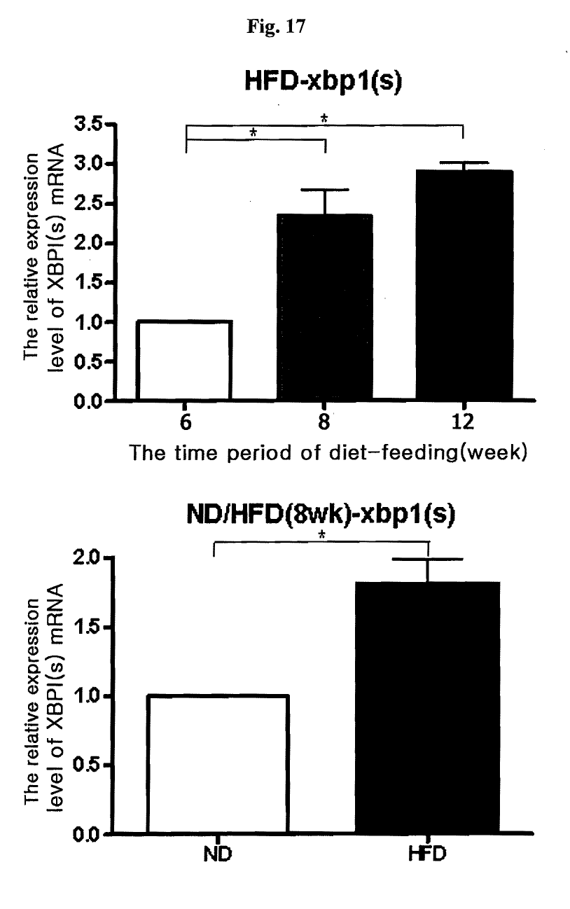

After being induced to be obese by feeding high fat diet (HFD) thereto, mice were measured for XBP1(S) mRNA expression level in adipose tissue (epididymal fat). For comparison, the adipose tissue from mice fed with normal diet (ND) was used as a control. Weights of HFD- and ND-fed mice are summarized in Table 1, below. Referring to FIG. 17, XBP1(S) mRNA expression levels in HFD-fed mice are plotted against the time period of HFD provision and a comparison in XBP1(S) mRNA expression level between ND- and HFD-fed mice on Week 8 is shown.

TABLE 1

| | Time Period of Feeding | | | | | |
|---|---|---|---|---|---|---|
| | Week 6 | | Week 8 | | Week 12 | |
| Mouse Model | ND | HD | ND | HD | ND | HD |
| Avg. Wt. (g) | 30.3 | 38.9 | 30.8 | 39.1 | — | 47.9 |

As is apparent from the data of Table 1, the HFD-fed mice significantly increased in weight compared to ND-fed mice. In addition, the histograms of FIG. 17 show an increase in the XBP1(S) mRNA expression level of HFD-fed mice in proportion to an increase in the weight thereof, with superiority to the XBP1(S) expression level of ND-fed mice.

### Example 7

### Effect of Rosiglitazone on the Differentiation of XBP1(S) Expression-Repressed Precursor Cells into Adipocytes

PPARγ2, a transcription factor known to induce adipocyte differentiation, is activated by rosiglitazone, an anti-diabetic drug. On the basis of the data showing that the expression level of PPARγ2 mRNA is decreased upon the differentiation of XBP1(S)-knock-down 3T3-L1 precursor cells (SFG-xbp1i) into adipocytes, an examination was made of whether the expression of PPARγ2 can be regulated by XBP1(S). In this regard, XBP1(S) expression-knock-down 3T3-L1 precursor cells (SFG-xbp1i) were induced to differentiate into adipocytes in the presence of 0 or 50 μM rosiglitazone in an induction medium during which the relative expression levels of PPARγ2 mRNA were measured.

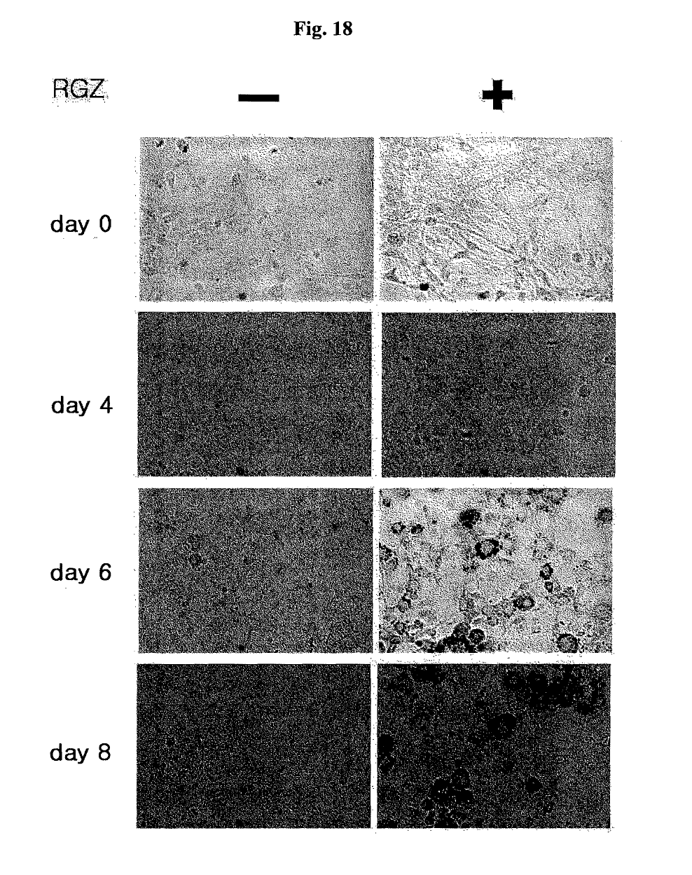

FIG. 18 shows how the rosiglitazone-induced increase of PPARγ2 mRNA expression level affects differentiation from XBP1(S)-knock-down 3T3-L1 precursor cells (SFG-xbp1i) to adipocytes in terms of morphology. As seen in FIG. 18, no adipocyte differentiation was observed in the cells in the absence of rosiglitazone (“−” panels) whereas mature adipocytes started to appear from Day 6 in the presence of 50 μM rosiglitazone (“+” panels).

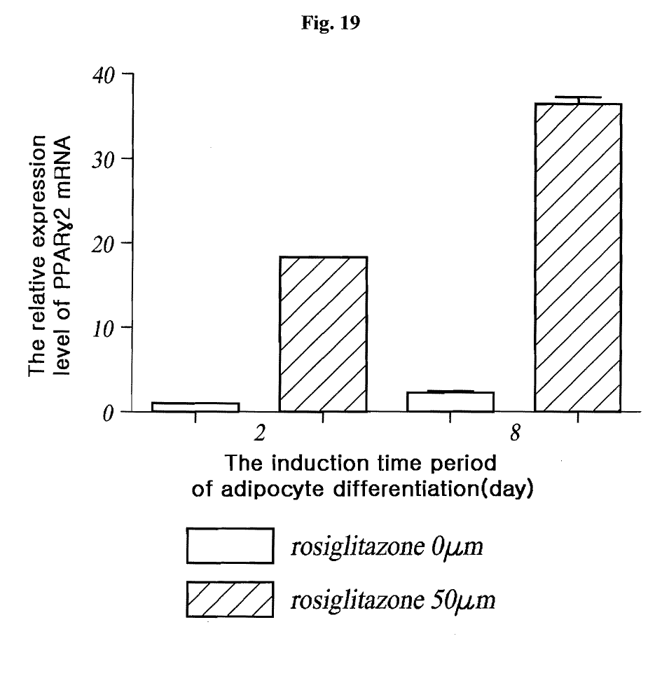

FIG. 19 shows the effect of rosiglitazone on the expression of PPARγ2 mRNA upon differentiation from XBP1(S)-knock-down 3T3-L1 precursor cells (SFG-xbp1i) to adipocytes. As is apparent from the data of FIG. 19, the expression level of PPARγ2 mRNA was 20 times as large in the presence of 50 μM rosiglitazone as in the absence of rosiglitazone, and increased in an induction time-dependent pattern.

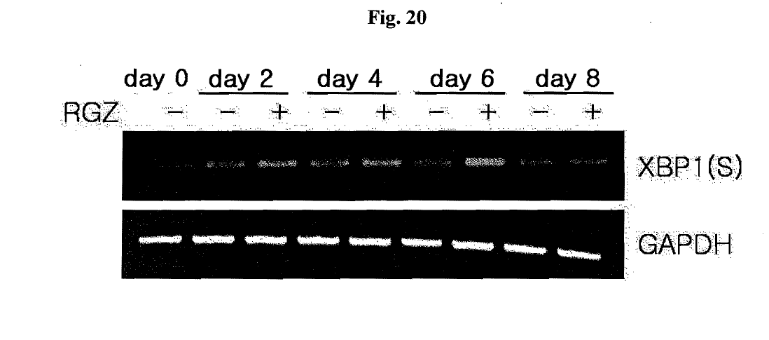

FIG. 20 shows the effect of rosiglitazone on the expression of XBP1(S) upon differentiation from XBP1(S)-knock-down 3T3-L1 precursor cells (SFG-xbp1i) to adipocytes. As seen in FIG. 20, the expression level of XBP1(S) was higher in the presence of 50 μM rosiglitazone (expressed by “+”) than in the absence of rosiglitazone (expressed by “−”) and increased in an induction time-dependent pattern.

Data obtained in Example 7 demonstrate that when applied to the treatment of diabetes, rosiglitazone induces the expression of XBP1(S) which in turn binds to the PPARγ2 promoter to promote adipocyte differentiation, resulting in obesity.

### INDUSTRIAL APPLICABILITY

When targeting the XBP1(S) gene or protein of the present invention, a compound featuring an ability to block or restrain differentiation into adipocytes can be used to develop an agent for the prevention and treatment of obesity.

1. A composition for measuring an increase in differentiation of a given cell into an adipocytes, comprising as an active ingredient an XBP1(S) {X box-binding protein 1, spliced form} having an amino acid sequence of SEQ ID NO. 1, 2 or 3, wherein said given cell is selected from the group consisting of a pre-adipocyte, an adipocyte precursor cell and a stem cell.

2. The composition of claim 1, wherein said given cell is derived from a mammal selected from a group consisting of a human, rat and mouse.

3. The composition of claim 1, wherein said XBP1(S) is proportional in mRNA expression level to that of an adipogenic marker selected from the group consisting of a PPARγ2 protein and an ap2 proteins.

4. The composition of claim 1, wherein said XBP1(S) is proportional in expression level to an XBP1(U) {X box-binding protein 1, unspliced form}.

5. The composition of claim 1, wherein said XBP1(S) functions to promote differentiation into adipocytes when increased in expression level.

6. The composition of claim 1, wherein said XBP1(S) functions to repress differentiation into adipocytes when decreased in expression level.

7. A promoter of adipocyte differentiation, comprising at least one molecule selected from the group consisting of an XBP1(S) protein and a polypeptide fragment having physiological activity identical to that of said XBP1(S) protein.

8. The promoter of claim 7, wherein said XBP1(S) protein has the amino acid sequence of SEQ ID NO. 1, 2 or 3.

9. A repressor of adipocyte differentiation, comprising an XBP1(S) expression inhibitor.

10. The repressor of claim 9, wherein said XBP1(S) protein has the amino acid sequence of SEQ ID NO. 1, 2 or 3.

11. The repressor of claim 9, wherein said XBP1(S) expression inhibitor is an siRNA (small interfering RNA) complementary to an mRNA of said XBP1(S).

12. The repressor of claim 11, wherein said siRNA is derived from an shRNA (small hairpin RNA).

13. A method for promoting adipocyte differentiation, said method comprising the steps of:
introducing into a given cell a promoter of adipocyte differentiation;
measuring a rate of differentiation into an adipocyte; and
further introducing said promoter of adipocyte differentiation when the rate of differentiation is lower than a predetermined standard,
wherein said given cell is selected from the group consisting of a pre-adipocyte, adipocyte precursor cell or stem cell, and
wherein said promoter comprises a molecule selected from the group consisting of an XBP1(S) protein, a polypeptide fragment having physiological activity identical to that of the XBP1(S) protein, and an XBP1(S) expression inducer.

14. The method of claim 13, wherein said XBP1(S) protein has the amino acid sequence of SEQ ID NO. 1, 2 or 3.

15. A method for repressing adipocyte differentiation, said method comprising the steps of:
introducing into given cell repressor of adipocyte differentiation, wherein said repressor comprises an XBP1(S) expression inhibitor;
measuring a rate of differentiation into an adipocyte; and
further introducing said repressor when the rate of differentiation is higher than a predetermined standard,
wherein said given cell is selected from the group consisting of a pre-adipocyte, an adipocyte precursor cell and a stem cell.

16. The method of claim 15, wherein said XBP1(S) protein has the amino acid sequence of SEQ ID NO. 1, 2 or 3.

17. The method of claim 15, wherein said rate measuring is carried out by an activity selected from the group consisting of morphologically observing differentiation into an adipocytes, determining an mRNA level of said XBP1(S), and determining an expression level of said XBP1(S).

18. A composition for screening a repressor of adipocyte differentiation, comprising an XBP1(S) gene.

19. The composition of claim 18, wherein said XBP1(S) gene comprises an mRNA or cDNA encoding the amino acid sequence of SEQ ID NO. 1, 2 or 3.

20. A composition for screening a repressor of adipocyte differentiation, comprising at least one molecule selected from the group consisting of an XBP1(S) protein and a polypeptide fragment having the same physiological activity as that of said XBP1(S) protein.

21. The composition of claim 20, wherein said XBP1(S) protein has the amino acid sequence of SEQ ID NO. 1, 2 or 3.

22. A method for screening a repressor of adipocyte differentiation, comprising:
bringing the agent of claim 18, comprising an XBP1(S) gene, into contact with a test material; and
determining whether said test material is promotive or repressive of XBP1(S) expression.

23. A method for screening a repressor of adipocyte differentiation, said method comprising:
bringing the agent of claim 20 into contact with a test material; and
determining whether said test material is promotive or repressive of the adipocyte differentiation inducing activity of a molecule selected from the group consisting of said XBP1(S) protein and said polypeptide fragment having the same physiological activity as that of said XBP1(S) protein.

24. A method for reducing rosiglitazone's side effect of causing obesity, said method comprising repressing XBP1(S) expression when said rosiglitazone is applied to treating diabetes.

25. A composition for predicting an increase in differentiation from given cell into an adipocytes, comprising as an active ingredient an XBP1(U) {X box-binding protein 1, unspliced form} having the amino acid sequence of SEQ ID NO. 4, 5 or 6,
wherein said given cell is selected from the group consisting of a pre-adipocyte, an adipocyte precursor cell or a stem cell.

26. The composition of claim 25, wherein said given cell is derived from a mammal selected from the group consisting of a human, rat and mouse.


SEQUENCE LISTING (6 sequences)

SEQ ID NO: 1 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 2 - protein, 371 aa, Rattus norvegicus
MVVVAAAPSAASAAPKVLLLSGQPASGGRALPLMVPGPRAAGSEASGTPQARKRQRLTHLSPEEKALRRKLKNRVAAQTARDRKKARMSELEQQVVDLEEENQKLQLENQLLREKTHGLVIENQELRTRLGMNALVTEEVSEAESKGNGVRLVAGSAESAAGAGPVVTSPEHLPMDSDAVASSDSESDILLGILDKLDPVMFFKCPSPESANLEELPEVYPEGPSSLPASLSLSVGTSSAKLEAINELIRFDHVYTKPLVLEIPSETESQTNVVVKIEEAPLSSSEEDHPEFIVSVKKEPLDDDFIPELGISNLLSSSHCLRPPSCLLDAHSDCGYEGSPSPFSDMSSPLGTDHSWEDTFANELFPQLISV

SEQ ID NO: 3 - protein, 371 aa, Mus musculus
MVVVAAAPSAATAAPKVLLLSGQPASGGRALPLMVPGPRAAGSEASGTPQARKRQRLTHLSPEEKALRRKLKNRVAAQTARDRKKARMSELEQQVVDLEEENHKLQLENQLLREKTHGLVVENQELRTRLGMDTLDPDEVPEVEAKGSGVRLVAGSAESAAGAGPVVTSPEHLPMDSDTVASSDSESDILLGILDKLDPVMFFKCPSPESASLEELPEVYPEGPSSLPASLSLSVGTSSAKLEAINELIRFDHVYTKPLVLEIPSETESQTNVVVKIEEAPLSSSEEDHPEFIVSVKKEPLEDDFIPELGISNLLSSSHCLRPPSCLLDAHSDCGYEGSPSPFSDMSSPLGTDHSWEDTFANELFPQLISV

SEQ ID NO: 4 - protein, 261 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 5 - protein, 267 aa, Rattus norvegicus
MVVVAAAPSAASAAPKVLLLSGQPASGGRALPLMVPGPRAAGSEASGTPQARKRQRLTHLSPEEKALRRKLKNRVAAQTARDRKKARMSELEQQVVDLEEENQKLQLENQLLREKTHGLVIENQELRTRLGMNALVTEEVSEAESKGNGVRLVAGSAESAALRLRAPLQQVQAQLSPPQNIFPWILTLLPLQILSLISFWAFWTSWTLSCFSNVLPQSLLIWRNSQRSTQKDLVPYQPPFLCQWGPHQPSWKPLMNSFVLTMYTPSL

SEQ ID NO: 6 - protein, 267 aa, Mus musculus
MVVVAAAPSAATAAPKVLLLSGQPASGGRALPLMVPGPRAAGSEASGTPQARKRQRLTHLSPEEKALRRKLKNRVAAQTARDRKKARMSELEQQVVDLEEENHKLQLENQLLREKTHGLVVENQELRTRLGMDTLDPDEVPEVEAKGSGVRLVAGSAESAALRLCAPLQQVQAQLSPPQNIFPWTLTLLPLQILSLISFWAFWTSWTLSCFSNVLPQSLLVWRNSQRSTQKDLVPYQPPFLCQWGPHQPSWKPLMNSFVLTMYTPSL